(19) United States
(12) Patent Application Publication
(10) Pub. No.: US 2016/0032233 A1
(43) Pub. Date: Feb. 4, 2016

(54) AERATION APPARATUS, AERATION METHOD AND CLEANING METHOD

(71) Applicant: CASHIDO CORPORATION, Miaoli County (TW)

(72) Inventors: SHIH-CHANG CHEN, MIAOLI COUNTY (TW); SHIH-MING CHEN, TAICHUNG CITY (TW); CHIH-HSIANG CHEN, MIAOLI COUNTY (TW); CHUN-LUNG CHIU, MIAOLI COUNTY (TW)

(21) Appl. No.: 14/450,223

(22) Filed: Aug. 2, 2014

(51) Int. Cl. C12M 1/00; B08B 3/08; B01F 3/04; C12M 1/34; C12M 1/02

(52) U.S. Cl. CPC C12M 29/24; C12M 41/40; C12M 39/00; C12M 27/00; B01F 3/04255; B08B 3/08

(57) ABSTRACT

An aeration apparatus, comprising: a gas-liquid mixing-circulating module, a gas supplying module, a controlling module and a cleaning unit, wherein the gas-liquid mixing-circulating module comprises: a first switch valve, connecting to a liquid outlet of a culture tank; a booster pump, connecting to the first switch valve; a micro-bubble producing device, having one end connecting to the booster pump and the other end connecting to a liquid inlet of the culture tank; a first proportional valve; a venturi tube, having a head, a tail and a branch, the other opening of the first proportional valve connecting to the head; and a second proportional valve, having an opening connecting to the tail, and the other opening connecting to the second tube, wherein, a fifth tube transfer gas to the venturi tube through the branch; wherein, the controlling module regulates the gas-liquid mixing-circulating module, the gas supplying module and the cleaning unit.

### BACKGROUND OF THE INVENTION

1. Field of the Invention

The present invention relates to an aeration apparatus, aeration method and cleaning method, in particular, to an aeration apparatus for supplying culture gas to a microorganism culture tank by producing micro bubbles and for regulating the amount of gas supply during the cultivation of the microorganism, an aeration method and cleaning method.

2. Description of Related Art

In the large-scale microorganism cultivation technique of the biological technology, the culture amount is calculated in metric tons, and for an extreme large amount of microorganism cultivation, in addition to a suitable medium, it is more important to maintain the environmental conditions suitable for the growth of microorganism to be constant, so that the metabolism, growth and other physiological status of the microorganism can be normally maintained, thereby achieving the high performance cultivation such as the fast growth rate and the high yield. If the growing environment of the microorganism is not properly controlled, it will result in the cultivation failure and thus a considerable loss for manufacturers.

Manufacturers have their own best experience in either the microorganism culture medium or the biological cultivation environment control. By taking an aerobic microorganism as an example, an aeration apparatus is one of the key factors that decide the growing rate of the microorganism. The number of the microorganism in any biological culture tank is too much to count, so providing enough oxygen by the aeration apparatus is quite crucial. Insufficient oxygen supply may result in an insufficient number of the microorganism or even its complete death, and total failure of cultivation may occur. In the above description, for a general aerobic microorganism, a safe content of oxygen in the culture solution (referred to as the culture medium) of the biological culture tank is a crucial factor. For example, in a 25-metric ton culture tank, since the number of the microorganism is considerable, the microorganism actually often stays in an environment of potential insufficient oxygen. Therefore, in order to meet the requirement for a large amount of oxygen consumption, a conventional safe practice in the prior art is to add an additional aeration in an amount of half of the culture tank volume, equivalent to injecting air at a rate of 12.5 metric tons per minute, which can just maintain the growth of this large number of the microorganism. However, in this case, bubble particles from the aeration in water are of a large size and are subject to relatively large buoyancy, so the bubbles can only stay in water for a short time, resulting in insufficient dissolved oxygen. Accordingly, an enough amount of the large bubbles must be supplied so as to compensate the dissolved oxygen. Hence, this method is less efficient and highly energy consuming, and the injected gas quickly rises to the top of the tank and is directly exhausted, so that the utilization of gas is low and only a limited amount of oxygen can be effectively dissolved in the culture solution.

Therefore, it is desirable in the art to improve the utilization of the gas injected into the biological culture tank, so as to save the energy consumption, reduce the cost and improve the production yield.

Accordingly, the invention that is reasonably designed and can effectively solve the above problems is proposed by the present inventors based on intensive researches and their knowledge.

### SUMMARY OF THE INVENTION

The object of the present invention is to provide an aeration apparatus, which is applied to a microorganism culture tank to solve the problem that a conventional microorganism culture tank is unable to efficiently obtain uniform gas supply for culture.

In order to achieve the aforementioned object, according to an embodiment of the present invention, an aeration apparatus is provided, for being installed in a microorganism culture tank to supply sterilized gas for microorganism cultivation and regulate the amount of gas supply, the aeration apparatus at least including: a gas-liquid mixing-circulating module, a gas supplying module and a controlling module. The gas-liquid mixing-circulating module includes: a first switch valve, connecting to a liquid outlet of the culture tank; a booster pump, connecting to the first switch valve through a first tube; at least one micro-bubble producing device, of which one end connects to the booster pump through a second tube, whereas of which the other end connects to a liquid inlet of the culture tank; a first proportional valve, of which an opening connects to the first tube by a third tube; a venturi tube, having a head, a tail and a branch, in which the other opening of the first proportional valve connects to the head; and a second proportional valve, of which an opening connects to the tail, whereas of which the other opening connects to the second tube by a fourth tube, wherein a fifth tube extends from the gas supplying module, the fifth tube connecting to the branch so that the gas supplying module transfers the gas to the venturi tube through the fifth tube. The controlling module at least electrically connects to the gas-liquid mixing-circulating module and the gas supplying module respectively, so as to regulate the micro-bubble producing device, the booster pump, the gas supplying module and the cleaning unit.

Preferably, according to the aeration apparatus of the present invention, the gas supplying module further includes a gas supplying unit. The gas supplying unit includes a third proportional valve and a second switch valve, of which an opening connects to the third proportional valve whereas of which the other opening connects to the branch through the fifth tube. The third proportional valve is operable to excessively capture the gas outside the aeration apparatus and transfer the gas to the second switch valve.

Preferably, according to the aeration apparatus of the present invention, the second switch valve connects to the third proportional valve through a relay pipe, and an exhaust pipeline extends from a middle part of the relay pipe, so that the relay pipe connects to an exhaust unit through the exhaust pipeline.

Preferably, according to the aeration apparatus of the present invention, the middle part of the relay pipe further includes a surge tank, and the surge tank further connects to the exhaust pipeline, the second switch valve and the third proportional valve respectively.

Preferably, according to the aeration apparatus of the present invention, the cleaning unit connects to a junction of the first tube and the third tube through a first cleaning pipe which is provided with a first cleaning valve; and the cleaning unit connects to the fourth tube through a second cleaning pipe which is provided with a second cleaning valve.

The present invention further provides an aeration method of an aeration apparatus, executable by the aforementioned aeration apparatus, which includes the following steps: closing the first cleaning valve and the second cleaning valve; providing a culture solution into the culture tank, in which the culture solution is a mixture of a microorganism and a culture medium; opening the first switch valve to release the culture solution to the booster pump; opening the booster pump to transfer the culture solution to the micro-bubble producing device; opening the first proportional valve, the second proportional valve, the third proportional valve and the second switch valve, so that the gas is transferred to the venturi tube by the gas supplying unit, and the second proportional valve extracts a part of the culture solution through the fourth tube to the venturi tube, and thus a gas-liquid mixing of the gas and the culture solution is carried out in the venturi tube, the culture solution then enters the first proportional valve and is transferred to the first tube through the third tube, and the booster pump transfers the culture solution to the micro-bubble producing device through the second tube; opening the micro-bubble producing device, so that after the culture solution passes through the micro-bubble producing device, the gas contained in the culture solution forms micro-bubbles in the culture solution; and transferring the culture solution through the micro-bubble producing device to the culture tank for the cultivation.

The present invention further provide a cleaning method of an aeration apparatus, executable by the aforementioned aeration apparatus, which includes the following steps: closing the first switch valve; closing the third proportional valve and the second switch valve, so as to disconnect the gas-liquid mixing-circulating module from the gas supplying unit; opening the booster pump, the first proportional valve, the second proportional valve and the micro-bubble producing device; opening the first cleaning valve and the second cleaning valve; and providing a cleaning solution, which is injected from the cleaning unit into the second tube and the fourth tube of the gas-liquid mixing-circulating module respectively through the first cleaning pipe and the second cleaning pipe, wherein the booster pump operates at a low rate between 100 rpm and 150 rpm; the first proportional valve, the second proportional valve and the micro-bubble producing device are full-bore opening, whereby the cleaning solution after cleaning the aeration apparatus is transferred to a drainage hole of the culture tank; and the culture tank further is provided with a steam inlet for injecting steam to sterilize the culture tank and the gas-liquid mixing-circulating module.

As above, the aeration apparatus of the present invention includes at least one micro-bubble producing device, so that 94% of the bubble particles are distributed between 0.2 μm and 0.3 μm (the test report is available in either Chinese or English version), and the bubbles are subjected to relatively low buoyancy and can stay in water for a longer time, thereby greatly increasing the dissolved oxygen. Thus, the gas supply efficiency for the growth of microorganisms is significantly improved, and the unnecessary consumption of energy sources is reduced. Moreover, the aeration method and the cleaning method facilitate the microorganism cultivation and reduce the possibility of biological cross contamination.

In order to further the understanding regarding the present invention, the following embodiments are provided along with illustrations to facilitate the disclosure of the present invention.

### BRIEF DESCRIPTION OF THE DRAWINGS

FIG. 1 shows a schematic functional block diagram of an aeration apparatus according to an embodiment of the present invention;

FIG. 2 shows a schematic view of pipelines of the aeration apparatus according to the embodiment of the present invention;

FIG. 3 shows a schematic flow chart of an aeration process executed by the aeration apparatus according to an embodiment of the present invention; and

FIG. 4 shows a schematic flow chart of a cleaning process executed by the aeration apparatus according to an embodiment of the present invention.

### DETAILED DESCRIPTION OF THE PREFERRED EMBODIMENTS

The aforementioned illustrations and following detailed descriptions are exemplary for the purpose of further explaining the scope of the present invention. Other objectives and advantages related to the present invention will be illustrated in the subsequent descriptions and appended drawings.

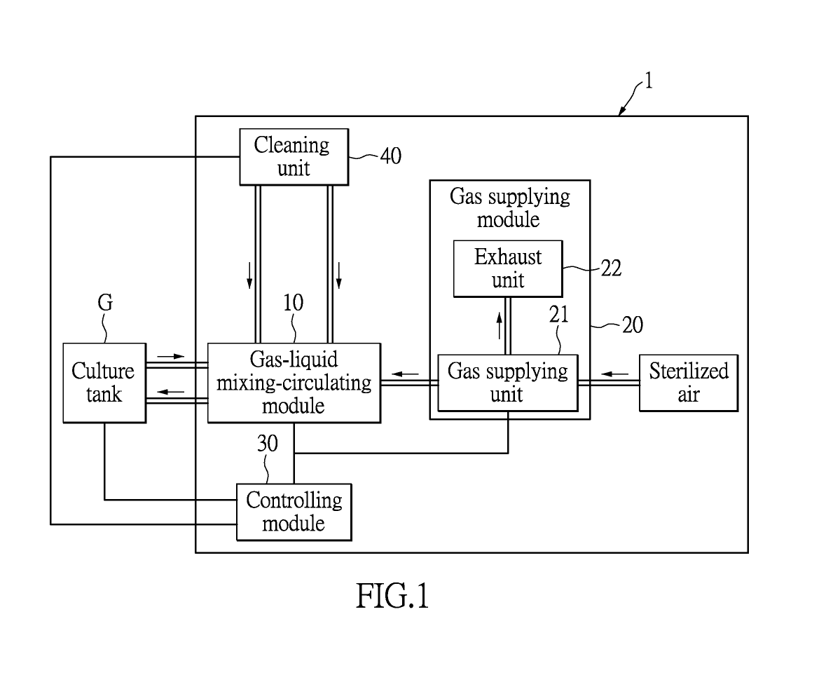

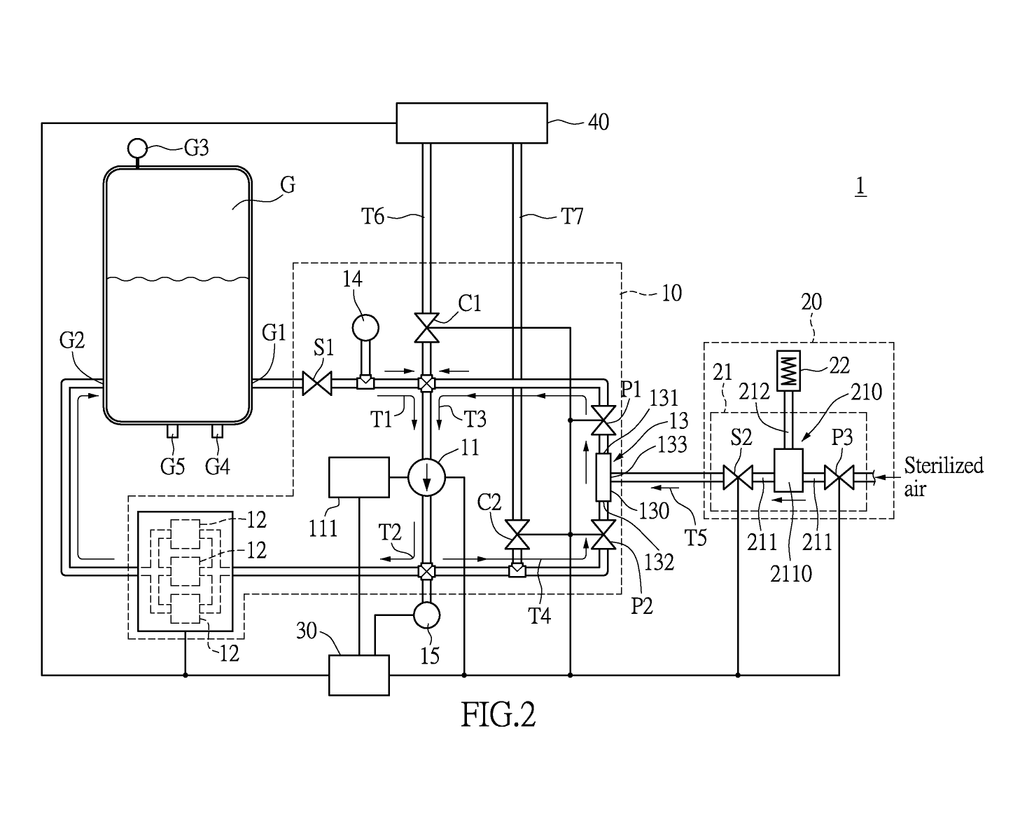

FIG. 1 shows a schematic functional block diagram of an aeration apparatus according to an embodiment of the present invention and FIG. 2 shows a schematic architectural view of the aeration apparatus according to the embodiment of the present invention. Referring to FIGS. 1 and 2, the present invention provides an aeration apparatus 1, for being installed in a microorganism culture tank G to supply gas for microorganism cultivation and regulate the amount of gas supply, in which the gas generally is a sterilized gas filtered through a filtration membrane of 0.1 μm (micrometer) to 0.45 μm. The aeration apparatus 1 at least includes: a gas-liquid mixing-circulating module 10, a gas supplying module 20, a controlling module 30 and a cleaning unit 40.

Further, the gas-liquid mixing-circulating module 10 at least includes: a first switch valve S1, a booster pump 11, at least one micro-bubble producing devices 12, a first proportional valve P1, a venturi tube 13 and a second proportional valve P2. The first switch valve S1 serves as a switch of a liquid outlet G1 of the culture tank G, and the booster pump 11 is located downstream relative to the first switch valve S1 and connects to the first switch valve S1 through a first tube T1.

The at least one micro-bubble producing devices 12 in this embodiment is preferably as shown in FIG. 2, in which 3 micro-bubble producing devices 12 are combined for example; however, the number is not limited thereto, and when the number of the micro-bubble producing devices 12 is only 1, it is already competent for the cultivation. It should be noted that when the number of the micro-bubble producing devices 12 is 2 or more, the amount of micro-bubbles for culture can be adjusted as required through the cooperation of the micro-bubble producing devices, so as to supply a possible large amount of gas for growth of the cultivated microorganism timely, which helps the adjustment of the gas supply margin. The microorganism culture in the present invention is a long-term continuous cultivation in most cases, and if more than one micro-bubble producing devices 12 are arranged, combined and used in the present invention, when one of the micro-bubble producing devices 12 needs maintenance or repair, another one of the micro-bubble producing devices 12 proceeds to execute the task to realize the continuous cultivation and maintain a good cultivation environment and status, thereby avoiding good culture status from turning to nothing. Additionally, the controlling module 30 electrically connects to the micro-bubble producing devices 12, so that the multiple micro-bubble producing devices 12, which operate either collaboratively or in turns in the multiplexing mechanism, all can be electrically connected to the controlling module 30 to achieve the purpose of regulation. However, it needs emphasizing that the number of the micro-bubble producing devices 12 is not limited to be 3 as described above.

Accordingly, the micro-bubble producing devices 12 are located downstream relative to the booster pump 11, in which one end of the micro-bubble producing devices 12 connects to the booster pump 11 through a second tube T2 whereas the other end of the micro-bubble producing devices 12 connects to a liquid inlet G2 of the culture tank G. In this manner, when the aeration is carried out for microorganism cultivation, the culture solution carrying the microorganism (the culture solution for short hereinafter) is drained out from the culture tank G via the liquid outlet G1, and in the meantime, the first switch valve S1 is opened to allow the culture solution to pass from the culture tank G to the first switch valve S1 via the liquid outlet G1 and then flow to the booster pump 11 through the first tube T1. An opening of the first proportional valve P1 connects to the first tube T1 through a third tube T3. The venturi tube 13 includes a front end 131, a rear end 132 and a branch 133, and the above first proportional valve P1 connects to the front end 131. An opening of the second proportional valve connects to the rear end 132 of the venturi tube 13, and the other opening connects to the second tube T2 through a fourth tube. Therefore, the culture solution after released through the first switch valve S1 flows to the booster pump 11 through the first tube T1, next flows to the micro-bubble producing devices 12 through the second tube T2, and then flows from the micro-bubble producing devices 12 to the liquid inlet G2 of the culture tank G. Moreover, based on the relationship between the first proportional valve P1 and the second proportional valve P2, by adjusting the revolutions for the two valves, a part of the culture solution is allowed to flow from the second tube T2 to the second proportional valve P2 via the fourth tube T4, then passes through the rear end 132 and the front end 131 of the venturi tube 13, and thereafter is transferred to the third tube T3 via the first proportional valve P1. In this manner, the culture solution then enters into the first tube T1 through the third tube T3.

A fifth tube T5 extends from the gas supplying module 20, and the fifth tube T5 connects to the branch 133 of the venturi tube 13. Therefore, the gas supplying module 20 is operable to transfer the gas (sterilized air) to the venturi tube 13 through the fifth tube T5, and thus inject the gas into the gas-liquid mixing-circulating module 10, in which the culture solution and the gas are primarily mixed in the venturi tube 13 of the gas-liquid mixing-circulating module 10. Since the front end 131 and the rear end 132 of the venturi tube 13 respectively connect to the first proportional valve P1 and the second proportional valve P2, after the revolutions for the first proportional valve P1 and the second proportional valve P2 are respectively adjusted, the partial flow rate of the culture solution between the front end 131 and the rear end 132 of the venturi tube 13 is increased, and based on the Bernoulli's principle, increasing the partial flow rate allows the gas to enter the venturi tube 13 through the branch 133 to be mixed with the culture solution. Therefore, the culture solution that reaches the third tube T3 via the front end 131 and the first proportional valve P1 is in the gas-liquid mixing status, and at the same time, after the culture solution returns from the third tube T3 to the booster pump 11 through the first tube T1, the booster pump 11 further helps to enhance the mixing of the gas and the culture solution, which is also a primary resource for achieving the sufficient mixing of the culture solution and the gas. Then, the culture solution can enter into the micro-bubble producing devices 12 through the second tube T2 (the former) or reach the second proportional valve P2 through the fourth tube T4 to enter into the venturi tube 13 again (the latter). For the former, the micro-bubble producing devices 12 compresses the sterilized air that has been mixed into the culture solution and the culture solution then flows to the culture tank G via the liquid inlet G2 of the culture tank G, so as to provide the oxygen required by the microorganism cultivation. For the latter, the culture solution that enters into the venturi tube 13 again can capture more sterilized air supplied from the gas supplying module 20 in the venturi tube 13. It's worthy of mentioning that the micro-bubble producing devices 12 convert the sterilized air carried in the culture solution into the micro-bubbles in the culture solution, and the volume of the micro-bubbles are extremely small and 94% of the micro-bubbles has a particle size in the range of 0.2 μm (micrometer) or 200 nm (nanometer) to 0.3 μm.

Compared with the bubbles having the smallest particle size of 1 mm (millimeter) available by the conventional aeration apparatus, the particle sizes of the micro-bubbles can reach 200 nm, and thus the micro-bubbles can stay in the culture tank for a longer period. In fact, the bubbles will continuously rise to the liquid surface due to the buoyancy, and in a liquid of a constant density, the buoyancy is directly proportional to the volume. In other words, the buoyancy of the bubbles in the liquid is directly proportional to the cube of radius of bubbles in the liquid. In addition, the large bubbles may join with other bubbles to be become larger in volume during continuous rising, and the rising speed of the bubbles in a liquid will be proportional to a variable greater than the cube of the radius. Therefore, the starting radius of the bubbles decides if the micro-bubbles can stay in the culture solution for a long period. For 200 nm bubbles, since the buoyancy is quite small, the kinetic energy of the bubbles in the water is relatively smaller, and all the bubbles suffer uniform pressure in the water and thus are not easy to join to each other. As such, oxygen can be more efficiently dissolved in the water to be used by the microorganism. Furthermore, such fine micro-bubbles promote the diffusion in the culture solution, which eliminates the dark corners of the gas supply in the culture tank. Additionally, the controlling module 30 at least electrically connects to the gas-liquid mixing-circulating module 10 and the gas supplying module 20 respectively, so as to regulate the overall action, on/off and the gas supply amount of the micro-bubble producing devices 12, the booster pump 11 and the gas supplying module 20.

Referring to FIGS. 1 and 2, the gas supplying module 20 can further include a gas supplying unit 21. The gas supplying unit 21 includes a third proportional valve P3 and a second switch valve S2 of which an opening connects to the third proportional valve P3 whereas of which the other opening connects to the branch 133 through the fifth tube T5. The third proportional valve P3 is operable to excessively capture the gas outside the aeration apparatus 1 and transfer the gas to the second switch valve S2. Preferably, the second switch valve S2 connects to the third proportional valve P3 through a relay pipe 210, and an exhaust pipeline 212 extends from a middle part 211 of the relay pipe 210 so that the relay pipe 210 connects to an exhaust unit 22 through the exhaust pipeline 212. Preferably, the middle part 211 of the relay pipe 210 further includes a surge tank 2110, in which the surge tank 2110 further respectively connects to the exhaust pipeline 212, the second switch valve S2 and the third proportional valve P3. In this case, the third proportional valve P3 can excessively capture the sterilized air from the outside, so that a positive pressure is formed inside the gas supplying unit 21 with the relay pipe 210 relative to the outside. Moreover, the exhaust pipeline 212 is connected to the exhaust unit 22 so that while the third proportional valve P3 excessively captures the sterilized air from the outside and transfers the sterilized air to the venturi tube 13 of the gas-liquid mixing-circulating module 10 via the second switch valve, the exhaust unit 22 releases the gas inside the gas supplying unit 21 to a reasonable extent, thereby avoiding too larger pressure in the gas supplying unit 21 to cause damage to parts and failures and the like. Therefore, the exhaust unit 22 is overall applicable to regulate the gas supplying unit 21 to maintain an appropriate relative positive pressure inside the gas supplying unit 21 to prevent the excessive pressure. Preferably, the exhaust unit 22 further includes a float gauge (not shown), for monitoring the positive pressure environment in the surge tank 2110 and even in the overall gas supplying unit 21. When the pressure in the gas supplying unit 21 is positive relative to the outside, which indicates that the gas supplying margin is sufficient and can be provided to the gas-liquid mixing-circulating module 10 any time through the branch 133 of the venturi tube 13, and the relative positive pressure can further effectively prevent bacteria from getting in from the outside to contaminate the microorganism. Therefore, a stable and uncontaminated sterilized air for the microorganism cultivation can be provided in the present invention, through the cooperation of the third proportional valve P3 of the gas supplying unit 21 and the exhaust unit 22. Incidentally, the second switch valve S2 is purely used as a switch herein, and in order to essentially create a positive pressure relative to the outside inside the gas supplying unit 21, the amount of gas captured by the third proportional valve P3 is at least larger than the total amount of gas exhausted by the exhaust unit 22 and the second switch valve S2. Furthermore, the operating speeds of the first proportional valve P1 and the second proportional valve S2 also influences the amount of gas exhausted by the second switch valve S2.

Therefore, more particularly, referring to FIGS. 1 and 2, the controlling module 30 electrically connects to the first proportional valve P1 to carry out parameters setting of the first proportional valve P1 and the second proportional valve P2 to determine the revolutions of the first proportional valve P1 and second proportional valve P2, and thus regulate the captured proportion of the sterilized air from the gas supplying unit 21 based on the Bernoulli's principle. Herein, the conditions for determining the revolutions of the first proportional valve P1 and the second proportional valve P2 primarily come from a pressure sensor G3 disposed in the culture tank G, in which the pressure sensor G3 is operable to monitor if the air pressure inside the culture tank G rises up or not. If the air pressure inside the culture tank G is in a descending status, a signal is sent to the controlling module 30, and the controlling module 30 then increases the revolutions of the first proportional valve P1 and the second proportional valve P2, that is, increases the transfer ratios of the first proportional valve P1 and the second proportional valve P2. The culture solution passes through the first proportional valve P1 and the second proportional valve P2 whose revolutions are appropriately adjusted, so that after a proper amount of the sterilized air is captured from the gas supplying unit 1, the culture solution and the sterilized air are sent to the first tube T1 through the third tube, then are intensively mixed by the booster pump 11, and transferred to the micro-bubble producing devices 12 and then returned to the culture tank G, thus finishing the aeration for further cultivation.

Moreover, preferably, a low pressure sensor 14 and a high pressure sensor 15 are further arranged upstream and downstream of the booster pump 11, i.e. the first tube T1 and the second tube T2 respectively, so as to monitor the pressures upstream and downstream of the booster pump 11. When the low pressure sensor 14 monitors a lower pressure value, it indicates that the revolution of the booster pump G may be too high which results in the pressure upstream of the first tube T1 is low, and in the meantime, the low pressure sensor 14 can return a signal to the controlling module 30 so as to reduce the revolution of the booster pump 11. The high pressure sensor 15 functions on the same principle. For example, when a higher pressure is monitored on the second tube T2, it indicates that too much liquid is accumulated before the micro-bubble producing devices 12, and the micro-bubble producing devices 12 may have the transfer pumping function. Herein, with the similar signal transmission mechanism described above, the controlling module 30 increases the revolution of the micro-bubble producing devices 12 or reduces the revolution of the booster pump G. The booster pump 11 is further externally connected with a frequency inverter 111, and in addition to the mechanism of variable revolutions, the frequency inverter 111 can achieve the purpose of saving energy. In this embodiment, the highest revolution of the booster pump 11 is up to 3600 rpm, and is not limited to this.

Incidentally, in the venturi tube 13, the tube between the front end 131 and the rear end 132 further constitutes a venturi tube body (not shown), the tube diameter of which is preferably larger than or equal to that of the fifth tube T5. The diameter of the venturi tube body 130 can also be smaller or equal to the fourth tube T4, the diameter of the fourth tube T4 can further be smaller than or equal to the third tube T3, the diameter of the third tube T3 is further smaller than or equal to the first tube T1, and the diameter of the first tube T1 is smaller than or equal to the second tube T2. As such, the diameters are substantially increasing, which allows the gas to be transferred to the culture tank G more easily, thereby increasing the gas supply efficiency.

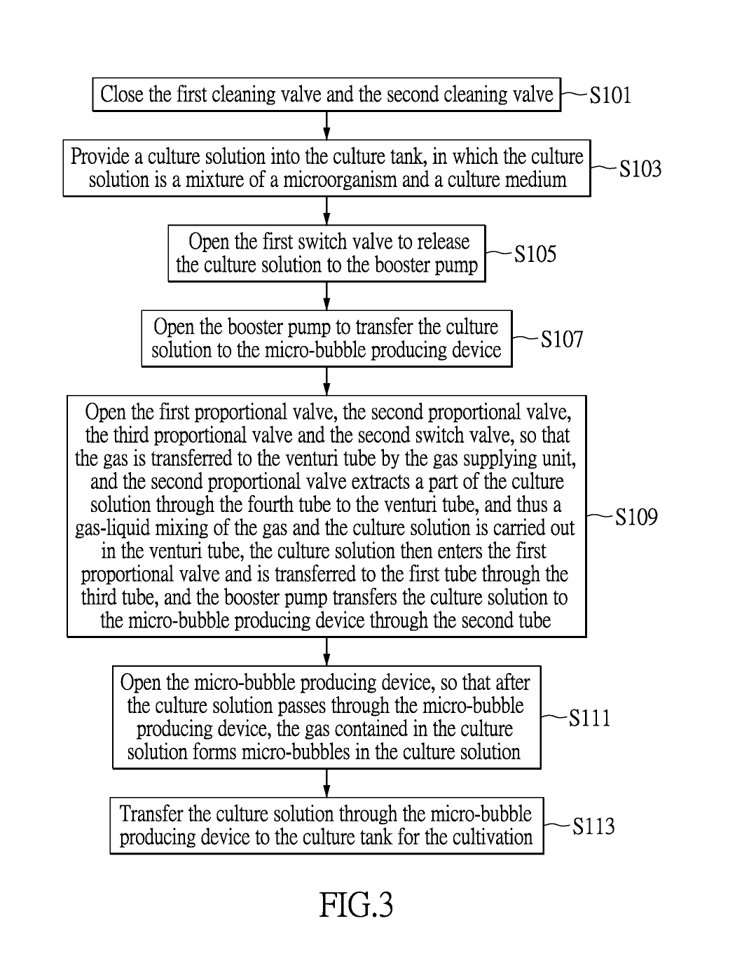

Referring to FIGS. 1, 2 and 3, in combination with the above-mentioned aeration apparatus 1, the present invention further provides an aeration method of the aeration apparatus, which includes the following steps: closing the first cleaning valve C1 and the second cleaning valve C2 (step S101); providing a culture solution (not shown) into the culture tank G, in which the culture solution is a mixture of a microorganism and a culture medium (step S103); opening the first switch valve S1 to release the culture solution to the booster pump 11 (step S105); opening the booster pump 11 so that the booster pump 11 transfers the culture solution to the micro-bubble producing devices 12 (step S107); sequentially opening the first proportional valve P1, the second proportional valve P2, the third proportional valve P3 and the second switch valve S2, so that the gas is transferred to the venturi tube 13 of the gas-liquid mixing-circulating module 10 by the gas supplying unit 21, in which the front end 131 and the rear end 132 of the venturi tube 13 further connect to the first proportional valve P1 and the second proportional valve P2 respectively, and the second proportional valve P2 is operable to extract a part of the culture solution from the second tube T2 through the fourth tube T4 that connects to the second tube T2, and owing to the pipeline arrangement between the second tube T2 and the fourth tube T4 and the second proportional valve P2, in the present invention, the second proportional valve P2 transfers the culture solution to the first proportional valve P1 through the venturi tube 13, thereby assisting the venturi tube 13 in capturing the gas through the branch 133 for the gas-liquid mixing of the culture solution and the gas, and therefore from now on, the culture solution contains the gas due to the gas-liquid mixing, the culture solution can then enter into the first proportional valve P1 and is transferred to and mixed in the first tube T1 through third tube T3, and thereafter, the booster pump 11 transfers the culture solution mixed with the gas to the micro-bubble producing devices 12 through the second tube T2 (step S109); opening the micro-bubble producing devices 12, so that after the culture solution mixed with the gas passes through the micro-bubble producing devices 12, the gas contained in the culture solution forms micro-bubbles in the culture solution (step S111); and further transferring the culture solution to culture tank G by the micro-bubble producing devices 12 for the cultivation (step S113).

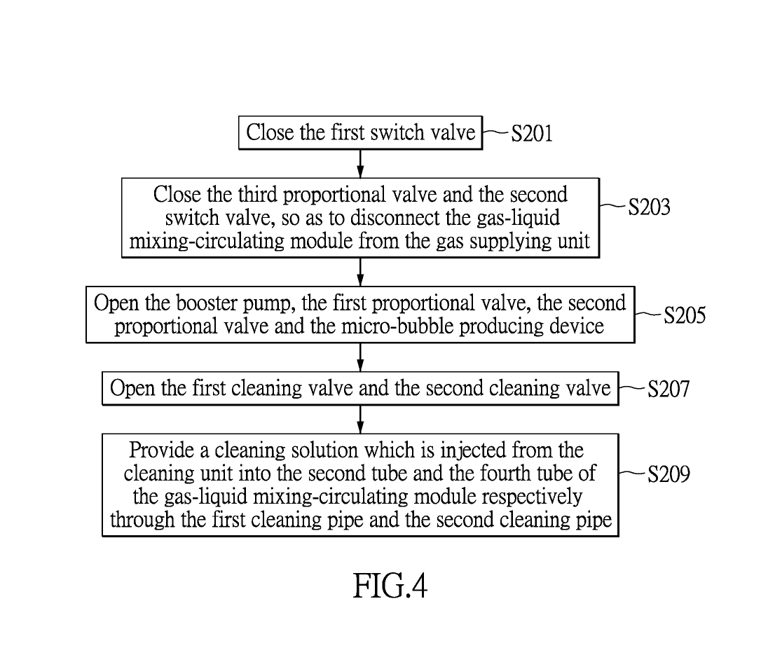

Referring to FIGS. 1, 2 and 4, in combination with the above-mentioned aeration apparatus 1, the present invention further provide a cleaning method of the aeration equipment, which includes the following steps: closing the first switch valve S1 (step S201); closing the third proportional valve P3 and the second switch valve S2, so as to disconnect the gas-liquid mixing-circulating module 10 from the gas supplying module 20 (step S203); opening the booster pump 11, the first proportional valve P1, the second proportional valve P2 and the micro-bubble producing devices 12 (step S205); opening the first cleaning valve C1 and the second cleaning valve C2 (step S207); and providing a cleaning solution (not shown) which is injected from the cleaning unit 40 into the junction of the second tube T2 and the third tube T3 and into the fourth tube T4 respectively through the first cleaning pipe T6 and the second cleaning pipe T7 (step S209). Herein, the booster pump 11 operates at a low rate set between 100 rpm and 150 rpm; the first proportional valve P1, the second proportional valve P2 and the micro-bubble producing devices 12 are full-bore opening, so that the cleaning solution can pass through all the pipes of the gas-liquid mixing-circulating module 10 smoothly, and thus the cleaning solution after cleaning the aeration apparatus 1 is efficiently transferred to a drainage hole G4 of the culture tank 11, in which the drainage hole G4 can also be used as a liquid collection hole for collecting the cultivated microorganism. Moreover, the culture tank G further is provided with a steam inlet G5 to achieve the requirements of the high-temperature and high-pressure sterilization in which the atmosphere is 1.2 atm and the temperature is 121° C. by injecting steam. As such, where the first switch valve S1 is opened, the first cleaning valve C1 and the second cleaning valve C2 are closed, and other settings remain unchanged and are identical to those of the above cleaning method, the steam can be injected via the steam inlet G5, so as to sterilize the culture tank G and even the overall gas-liquid mixing-circulating module 10. Therefore, the sterilization method of the present invention further includes providing a high-temperature and high-pressure steam sterilization effect.

In summary, the aeration apparatus 1 of the present invention can effectively solve the problem of inefficient gas supply and further reduce the cost of energy sources. For example, for cultivating 20 metric tons of Ganoderma (lingzhi) mycelium, the conventional aeration apparatus needs to consume 75 kW electricity per hour, equivalent to 1800 kW a day, to produce 10 metric tons of aeration per minute. By comparison, under the same culture specification conditions, the present invention only consumes 30 kW electricity per hour, equivalent to 720 kW a day, which saves the energy by over 50%. Furthermore, the aeration method derived from the aeration apparatus of the present invention adopts the operation control of a plurality of valves, which efficiently promotes the mixing of the culture solution and the gas and significantly improves the working efficiency of the micro-bubble producing apparatus contained in the present invention. Obviously, in addition to fungus such as Ganoderma (lingzhi), the present invention is also applicable to the cultivation of other types of microorganisms, including bacteria or algae, and can supply a gas different from air or oxygen as only as it is one required for the growth of a microorganism. In addition, the abovementioned cleaning and steam sterilization methods can provide effective equipment maintenance and eliminate contamination sources, thereby avoiding the potential cultivation failure.

The descriptions illustrated supra set forth simply the preferred embodiments of the present invention; however, the characteristics of the present invention are by no means restricted thereto. All changes, alternations, or modifications conveniently considered by those skilled in the art are deemed to be encompassed within the scope of the present invention delineated by the following claims.

What is claimed is:

1. An aeration apparatus, for being installed in a microorganism culture tank to supply sterilized gas for microorganism cultivation and regulate the amount of gas supply, the aeration apparatus at least comprising:
a gas-liquid mixing-circulating module, a gas supplying module, a controlling module and a cleaning unit, wherein
the gas-liquid mixing-circulating module comprises:
a first switch valve, connecting to a liquid outlet of the culture tank;
a booster pump, connecting to the first switch valve through a first tube;
at least one micro-bubble producing device, of which one end connects to the booster pump through a second tube, whereas of which the other end connects to a liquid inlet of the culture tank;
a first proportional valve, of which an opening connects to the first tube through a third tube;
a venturi tube, having a head, a tail and a branch, the other opening of the first proportional valve connecting to the head; and
a second proportional valve, of which an opening connects to the tail, whereas of which the other opening connects to the second tube through a fourth tube, wherein,
a fifth tube extends from the gas supplying module, the fifth tube connecting to the branch, so that the gas supplying module is operable to transfer the gas to the venturi tube through the fifth tube;
the controlling module at least electrically connects to the gas-liquid mixing-circulating module, the gas supplying module and the cleaning unit, respectively, to regulate the micro-bubble producing device, the booster pump, the gas supplying module and the cleaning unit.

2. The aeration apparatus according to claim 1, wherein the gas supplying module further comprises a gas supplying unit, wherein, the gas supplying unit comprises a third proportional valve and a second switch valve of which an opening connects to the third proportional valve, whereas of which the other opening connects to the branch through the fifth tube, and the third proportional valve is operable to excessively capture the gas outside the aeration apparatus and transfer the gas to the second switch valve.

3. The aeration apparatus according to claim 2, wherein the second switch valve connects to the third proportional valve through a relay pipe, and an exhaust pipeline extends from a middle part of the relay pipe, the relay pipe connecting to an exhaust unit through the exhaust pipeline.

4. The aeration apparatus according to claim 3, wherein the middle part of the relay pipe further includes a surge tank, the surge tank further connecting to the exhaust pipeline, the second switch valve and the third proportional valve respectively.

5. The aeration apparatus according to claim 4, wherein the cleaning unit connects to a junction of the first tube and the third tube through a first cleaning pipe, the first cleaning pipe being provided with a first cleaning valve; and the cleaning unit connects to the fourth tube through a second cleaning pipe, the second cleaning pipe being provided with a second cleaning valve.

6. The aeration apparatus according to claim 1, wherein the first tube is further provided with a low pressure sensor; and the second tube is further provided with a high pressure sensor.

7. The aeration apparatus according to claim 5, wherein the third proportional valve is operable to form a relative positive pressure inside the gas supplying module relative to the outside.

8. The aeration apparatus according to claim 5, wherein the third proportional valve, the second switch valve, the surge tank and the exhaust unit are operable to collaboratively regulate the gas supply amount to supply the gas to the gas-liquid mixing-circulating module.

9. The aeration apparatus according to claim 7, wherein the third proportional valve, the second switch valve, the surge tank and the exhaust unit are operable to collaboratively regulate the gas supply amount to supply the gas to the gas-liquid mixing-circulating module.

10. An aeration method of an aeration apparatus, executable by the aeration apparatus according to claim 8, comprising the steps of:
closing the first cleaning valve and the second cleaning valve;
providing a culture solution into the culture tank, the culture solution being a mixture of a microorganism and a culture medium;
opening the first switch valve to release the culture solution to the booster pump;
opening the booster pump to transfer the culture solution to the micro-bubble producing device;
opening the first proportional valve, the second proportional valve, the third proportional valve and the second switch valve, so that the gas is transferred to the venturi tube by the gas supplying unit, and the second proportional valve extracts a part of the culture solution through the fourth tube to the venturi tube, and thus a gas-liquid mixing of the gas and the culture solution is carried out in the venturi tube, the culture solution then enters the first proportional valve and is transferred to the first tube through the third tube, and the booster pump transfers the culture solution to the micro-bubble producing device through the second tube;
opening the micro-bubble producing device, so that after the culture solution passes through the micro-bubble producing device, the gas contained in the culture solution forms micro-bubbles in the culture solution; and
transferring the culture solution to the culture tank by the micro-bubble producing device for the cultivation.

11. A cleaning method of an aeration apparatus, executable by the aeration apparatus according to claim 8, comprising the steps of:
closing the first switch valve;
closing the third proportional valve and the second switch valve, so as to disconnect the gas-liquid mixing-circulating module from the gas supplying unit;
opening the booster pump, the first proportional valve, the second proportional valve and the micro-bubble producing device;
opening the first cleaning valve and the second cleaning valve; and
providing a cleaning solution which is injected from the cleaning unit into the second tube and the fourth tube of the gas-liquid mixing-circulating module respectively through the first cleaning pipe and the second cleaning pipe, wherein,
the booster pump operates at a low rate set between 100 rpm and 150 rpm;
the first proportional valve, the second proportional valve and the micro-bubble producing device are full-bore opening, whereby the cleaning solution after cleaning the aeration apparatus is transferred to a drainage hole of the culture tank and the culture tank further is provided with a steam inlet for injecting steam to sterilize the culture tank and the gas-liquid mixing-circulating module.
